(19) United States
(12) Patent Application Publication
(10) Pub. No.: US 2013/0138036 A1
(43) Pub. Date: May 30, 2013

(54) SURGICAL IMPLEMENTS WITH SUCTION AND IRRIGATION PASSAGES

(71) Applicants: Clifford T. Solomon, Severna Park, MD (US); Theodore C. Solomon, Hampstead, MD (US)

(72) Inventors: Clifford T. Solomon, Severna Park, MD (US); Theodore C. Solomon, Hampstead, MD (US)

(21) Appl. No.: 13/640,309

(22) PCT Filed: Mar. 28, 2011
(86) PCT No.: PCT/US2011/030189

§ 371 Date: Feb. 6, 2013

Related U.S. Application Data
(60) Provisional application No. 61/282,837, filed on Apr. 7, 2010.

(51) Int. Cl. A61M 3/02

(52) U.S. Cl. CPC A61M 3/0283
USPC 604/43

(57) ABSTRACT

The surgical implements (10) with suction and irrigation passages (28a, 28b) each include separate suction and irrigation passages disposed internally and formed integrally within the shank (12) or shaft of the implement. The implement may be a periosteal elevator (10) having a relatively wide and thin tip (18). The two passages (28a, 28b) extend from the handle (14) end to a common plenum (26) within the tip portion (18), with a series of common passages (30) extending from the plenum (26) to the distal edge (20) of the tip (18). Alternatively, the implement might be a nerve root retractor (110) having a relatively narrow tip (118) with suction and irrigation passages (128a, 128b) extending from the handle (114) end through the shank (112) of the device, the passages (128a, 128b) remaining separated from one another. The shank (12, 112) of the surgical implements may be straight or may have a bend ranging from 0° to 90°.

### TECHNICAL FIELD

The present invention relates generally to surgical tools and implements, and particularly to surgical implements with suction and irrigation passages for irrigation and/or suction of the wound during surgery.

### BACKGROUND ART

When performing surgery, it is often necessary to withdraw extraneous fluids (blood, etc.) from the surgical wound in order to gain a clear view of the surgical site. It is also necessary from time to time to flush debris (clotted blood, tissue fragments, etc.) from the site for the same reason, and/or to avoid contamination of the site. These needs apply to most surgeries, with a prime example being in the field of spinal surgery.

As a result, a number of devices have been developed for the purposes of providing suction to a surgical wound and irrigating the wound. Generally, these devices are single purpose, i.e., they cannot perform surgical tasks or functions other than suction or irrigation. Certain other implements or instruments have been developed with suction and/or irrigation passages, but at least one of the passages in these devices is externally disposed to the shank or shaft of the instrument.

Thus, surgical implements with suction and irrigation passages solving the aforementioned problems are desired.

### DISCLOSURE OF INVENTION

The surgical implements with suction and irrigation passages include separate suction and irrigation passages disposed internally, within the shank or shaft of the instrument. One embodiment comprises a periosteal elevator, i.e., a surgical tool for lifting (elevating) the periosteal tissue layer from the underlying bone. The periosteal elevator includes separate suction and irrigation passages disposed internally and integrally within the shank or shaft of the instrument, extending from the handle end to the opposite distal working end. The two separate passages may terminate in a common plenum disposed within the tip portion of the device, with a series of short common passages extending from the plenum to the tip.

A second embodiment comprises a nerve root retractor having a narrower tip than the periosteal elevator. The nerve root retractor also includes separate suction and irrigation passages extending through the length of the shank of the tool and formed internally and integrally therewith. However, the narrow tip of the nerve root retractor precludes a plenum and common passages extending therefrom. In the nerve root retractor embodiment, each of the suction and irrigation passages extends completely to the tip of the instrument, with the two passages remaining separate and distinct from one another.

The specific configuration of the tip of the instrument, as well as the handle or shank, may be adjusted or configured as desired, with the common theme of all of the instruments being the separate passages extending through the instruments to provide suction and irrigation during surgical procedures. The shank of either tool, and particularly the nerve root retractor, may be straight or may include an angle, preferably between 0 and 90 degrees. The instruments may be formed of material adapted for reuse after sterilization, e.g., surgical stainless steel, or may alternatively be formed of materials providing economical disposal, e.g., certain plastics, carbon fiber, etc.

These and other features of the present invention will become readily apparent upon further review of the following specification and drawings.

### BRIEF DESCRIPTION OF DRAWINGS

FIG. 1 is a perspective view of a periosteal elevator surgical implement according to the present invention having both suction and irrigation passages therein.

FIG. 2 is an enlarged top plan view of the distal tip of the surgical implement of FIG. 1, showing further details thereof.

FIG. 3 is an enlarged side elevation view of the distal shank and tip of the surgical implement of FIG. 1, showing further details thereof.

FIG. 4 is an enlarged end elevation view of the distal tip of the surgical implement of FIG. 1, showing further details thereof.

FIG. 5 is a perspective view of a nerve root retractor surgical implement according to the present invention having both suction and irrigation passages therein.

FIG. 6 is a broken away perspective view of an alternate embodiment of a nerve root retractor according to the present invention having a 90° angle stem and tip.

FIG. 7 is a broken away perspective view of an alternate embodiment nerve root retractor according to the present invention having a 30° angle stem and flanged tip.

FIG. 8 is a perspective view of another alternative embodiment of a nerve root retractor according to the present invention having an angular offset in the stem or shank thereof, and separate suction and irrigation passages extending to the tip.

Similar reference characters denote corresponding features consistently throughout the attached drawings.

### BEST MODE FOR CARRYING OUT THE INVENTION

The present invention comprises various embodiments of surgical implements having separate suction and irrigation passages disposed integrally within their shanks, and extending from the handle ends to the distal tips of the tools. These implements or instruments are thus capable of withdrawing debris from a surgical wound and also irrigating the wound as desired, without need for additional surgical devices.

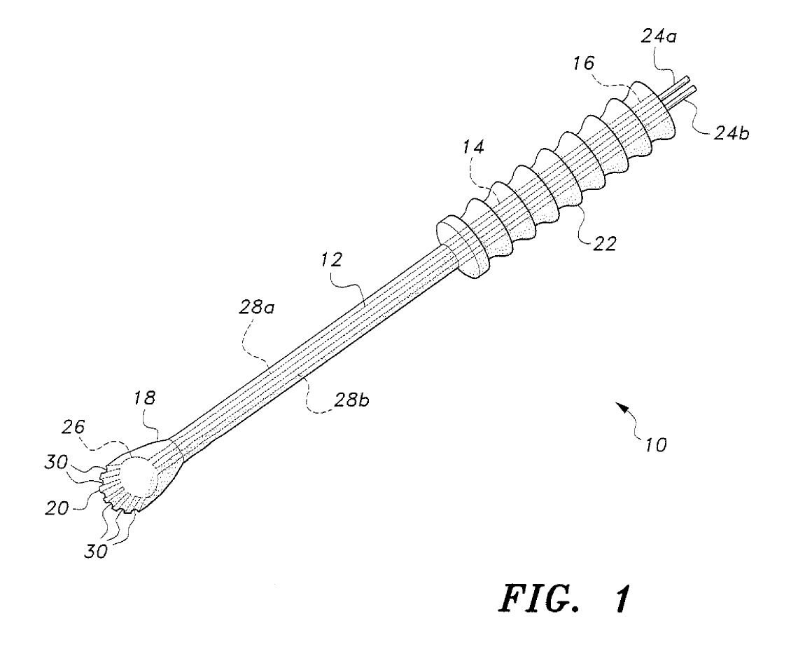

FIG. 1 of the drawings illustrates a periosteal elevator 10, i.e., a surgical implement for lifting or elevating the connective tissue immediately adjacent the bone. The implement or tool 10 comprises an elongate shank 12 having a handle portion 14 and proximal end 16, and a distal tip portion 18 with distal inlet and outlet end 20 opposite the handle portion 14 and end 16. The shank 12 may be formed of surgical grade corrosion resistant steel, i.e., “stainless steel,” with the handle portion 14 preferably including a handle 22 disposed thereon. Alternatively, the shank 12 including its tip 18 may be formed of economically disposable materials, such as certain plastics, carbon fiber, etc.

Two passages are formed integrally within the shank 12, and extend from corresponding inlet and outlet supply tubes or fittings 24a and 24b at the proximal end 16 of the device to a plenum 26 disposed within the distal tip portion 18. These passages comprise an irrigation passage 28a for providing a fluid for flushing the wound, and a suction passage 28b for withdrawing excessive fluid and debris from the wound during surgery. It should be noted that while the two separate passages are indicated specifically as irrigation passage 28a and suction passage 28b in the drawing Figs., they may be interchanged functionally as desired.

A series of small, distal combination suction and irrigation passages 30 extend from the plenum 26 to the distal inlet and outlet end 20 of the tip portion 18 of the device. These suction and irrigation passages 30 communicate with the plenum 26, which in turn communicates with the two passages 28a and 28b extending through the shank 12 to the inlet and outlet fittings 24a and 24b. Thus, irrigation fluid applied through the inlet fitting 24a passes through the irrigation passage 28a, plenum 26, and outwardly through the plurality of distal combination irrigation and suction passages 30. Conversely, suction applied to the outlet fitting 24b is transmitted through the suction passage 28b to the plenum 26, and thence to the wound via the combination passages 30 extending to the distal inlet and outlet end 20 of the device. While there is some mixing of withdrawn and irrigation fluids within the combination passages and plenum, it will be seen that the volume is quite small, on the order of a small fraction of a milliliter, and thus of no practicable consequence.

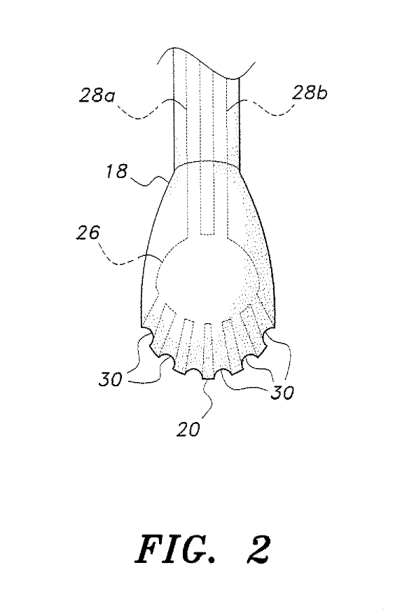

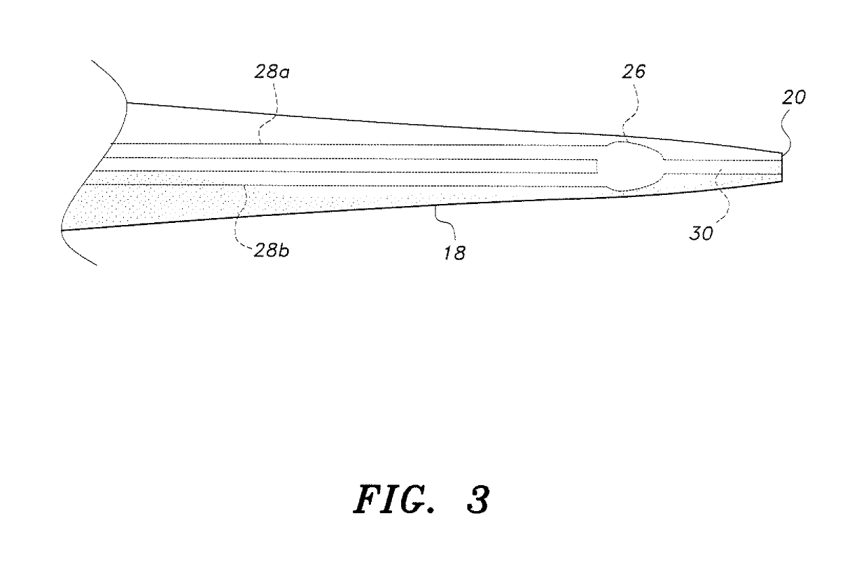

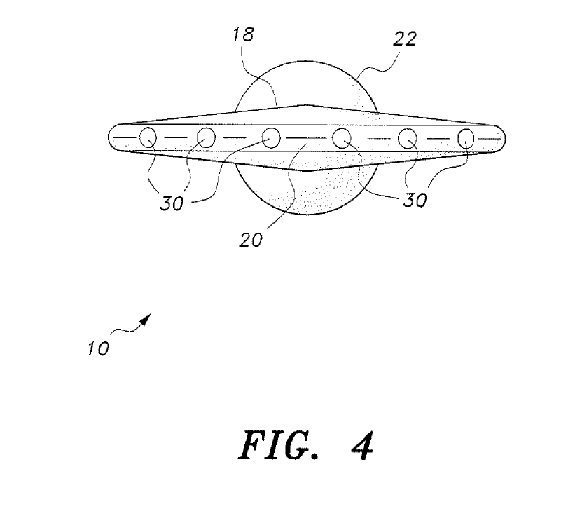

FIGS. 2 through 4 provide more detailed views of the tip portion 18 of the periosteal elevator 10. The distal tip portion 18 of the device is preferably somewhat more broad and thin than the shank 12, as shown particularly in FIGS. 2 through 4, This enables the tip portion 18 to be used as a scraping, prying, and lifting/elevating instrument for separation of the overlying periosteal connective tissue from the underlying bone, as is often required in certain types of surgery. Yet, the two irrigation and suction passages 28a, 28b extending through the instrument 10 enable the surgeon to apply either irrigation or suction to the surgical wound as desired, without need for a separate device. This greatly facilitates the surgeon's task, by avoiding the introduction and complication of an additional tool or tools into the wound.

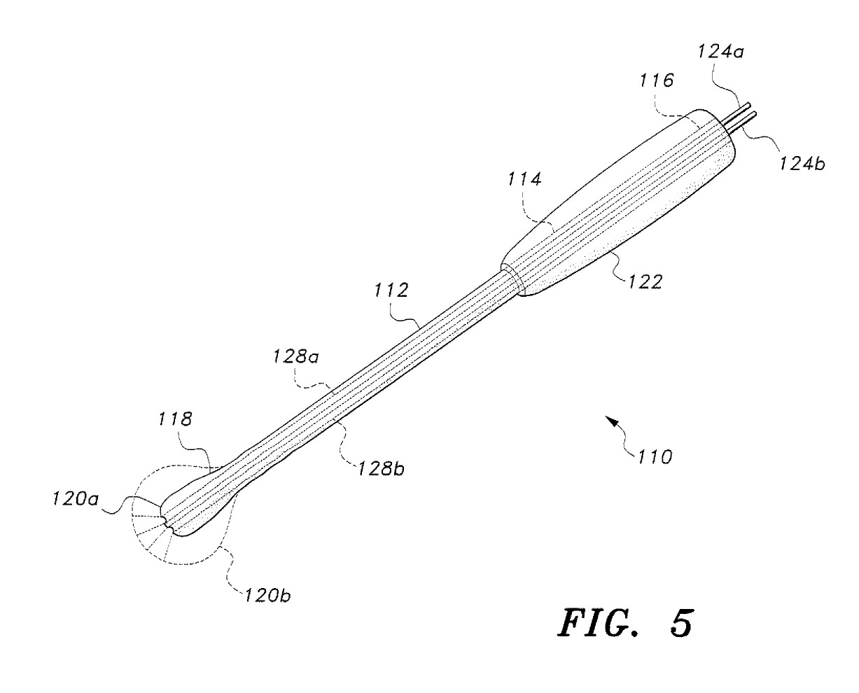

FIGS. 5 through 8 illustrate various embodiments of a nerve root retractor implement including irrigation and suction passages therein. The nerve root retractors 110, 210, 310, and 410 respectively of FIGS. 5 through 8 have somewhat narrower shanks than the periosteal elevator 10 of FIGS. 1 through 4, and thus the tips are not widened or flattened to such an extent. Some widening and/or flattening may be provided, depending upon the desired shape of the tip of the tool.

The nerve root retractor 110 of FIG. 5 is configured much like the periosteal elevator 10 of FIGS. 1 through 4, comprising an elongate shank 112 having a handle portion 114 and proximal end 116, and a distal tip portion 118 with distal inlet and outlet end opposite the handle portion 114 and end 116. A relatively narrow end 120a is illustrated in solid lines for the surgical implement 110 of FIG. 5, with an alternative wider end 120b being shown in broken lines. It will be appreciated that myriad other shapes and sizes for the distal end of the implement 110 may be provided as desired so long as the implement contains two separate passages therethrough for suction and irrigation, in keeping with other embodiments disclosed herein.

As in the case of the periosteal elevator 10 of FIGS. 1 through 4, the shank 112 of the nerve root retractor 110 is straight and devoid of bends. The shank 112 may be formed of surgical grade corrosion resistant steel, i.e., “stainless steel,” or may alternatively be formed of an economically disposable material as noted further above. The handle portion 114 preferably includes a handle 122 disposed thereon, as in the case of the periosteal elevator 10. Two passages are formed integrally within the shank 112, and extend from corresponding inlet and outlet supply tubes or fittings 124a and 124b at the proximal end 116 of the device to the distal inlet and outlet end 120a or 120b. These passages comprise an irrigation passage 128a for providing a fluid for flushing the wound, and a suction passage 128b for withdrawing excessive fluid and debris from the wound during surgery. It should be noted that while the two separate passages are indicated specifically as irrigation passage 128a and suction passage 128b in FIG. 5, they may be interchanged functionally as desired.

The tip portion 118 of the nerve root retractor 110 is relatively small and narrow for the inclusion of a plenum and several common irrigation and suction passages therein. Accordingly, the two passages 128a and 128b extend through the shank 112 from the proximal end 116 of the device to the distal inlet and outlet end 120a or 120b, and no communication takes place between the two passages. Thus, irrigation fluid applied through the inlet fitting 124a passes through the irrigation passage 128a and directly outwardly through the outlet end of the passage 128a at the distal end 120a or 120b of the tip. Conversely, suction applied to the outlet fitting 124b is transmitted through the suction passage 128b directly to the inlet end of the passage 128b at the distal tip end 120a or 120b, and thence to the wound. As the two passages 128a and 128b are completely separated throughout the extent of the nerve root retractor 110, there is no mixing of fluids between the inlet and outlet passages of the device.

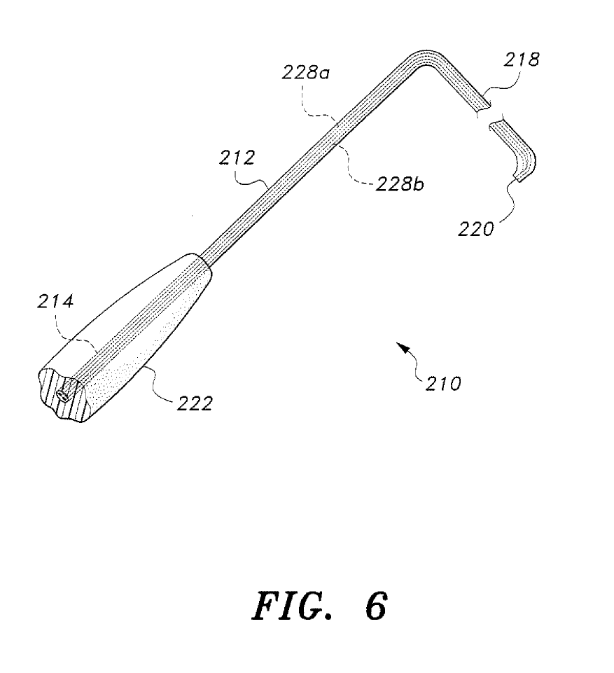

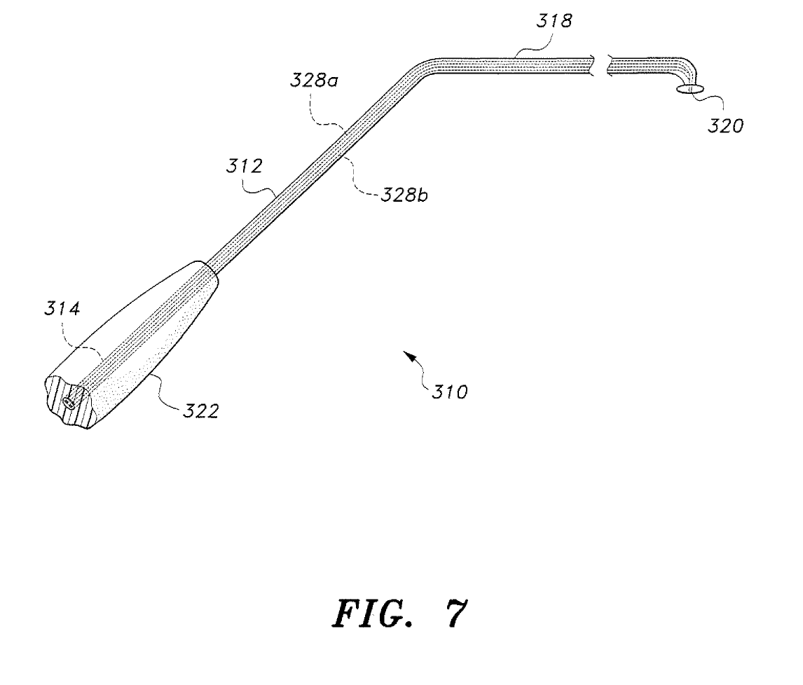

FIGS. 6 and 7 illustrate the critical features of a pair of alternate embodiment nerve root retractors, respectively designated as retractors 210 and 310. The nerve root retractor 210 of FIG. 6 includes an elongate shank 212 having a handle portion 214 and opposite distal tip portion 218 and distal inlet and outlet end of the tip 220, with the shank 212 and its distal tip portion 218 having essentially the same diameter and cross-sectional shape or configuration from the handle portion 214 to a point very near the distal tip 220. The handle portion 214 of the shank 212 includes a handle 222 thereon. Separate irrigation and suction passages, respectively 228a and 228b, extend through the length of the shank 212, as in the case of the retractor 110 of FIG. 5. The irrigation and suction passages 228a, 228b enter the instrument 210 through separate inlet and outlet fittings (not shown in FIG. 6), essentially as in the instrument 110 of FIG. 5. The primary difference between the retractor 210 of FIG. 6 and the retractor 110 of FIG. 5 is that the shank 212 is bent, with the tip portion 218 of the retractor 210 forming an angle of substantially 90 degrees relative to the major portion of the shank 212. The distal inlet and outlet end 220 of the tip portion 218 may be bent through another 90 degrees if so desired, generally as shown in FIG. 6. This can facilitate certain procedures using the surgical instrument 210.

FIG. 7 illustrates a nerve root retractor 310 having a configuration much like that of the retractor 210 of FIG. 6. The retractor instrument 310 of FIG. 7 includes an elongate shank 312 having a handle portion 314 and opposite distal tip portion 318 and distal inlet and outlet end of the tip 320, with the shank 312 and its distal tip portion 318 having essentially the same diameter and cross-sectional shape or configuration from the handle portion 314 to the distal tip 320. The handle portion 314 of the shank 312 includes a handle 322 thereon. Separate irrigation and suction passages, respectively 328a and 328b, extend through the length of the shank 312, as in the cases of the retractors 110 and 210 respectively of FIGS. 5 and 6. The irrigation and suction passages 328a, 328b enter the instrument 310 through separate inlet and outlet fittings (not shown in FIG. 7), essentially as in the instrument 110 of FIG. 5. The primary difference between the retractor 310 of FIG. 7 and the retractor 210 of FIG. 6 is that the shank 312 is bent to form an angle of substantially 30 degrees relative to the major portion of the shank 312. It will be seen that any angle desired may be formed in the shank of the nerve root retractor of the present invention, with the 90 degree angle of the instrument of FIG. 6 and the 30 degree angle of the instrument of FIG. 7 being exemplary but not limiting. The extreme distal inlet and outlet end 320 of the surgical implement 310 of FIG. 7 will also be seen to differ somewhat from other embodiments by having a small flange extending outwardly from the inlet and outlet passages, with the plane of the flange being at least generally normal to the axes of the inlet and outlet passages at their most distal points.

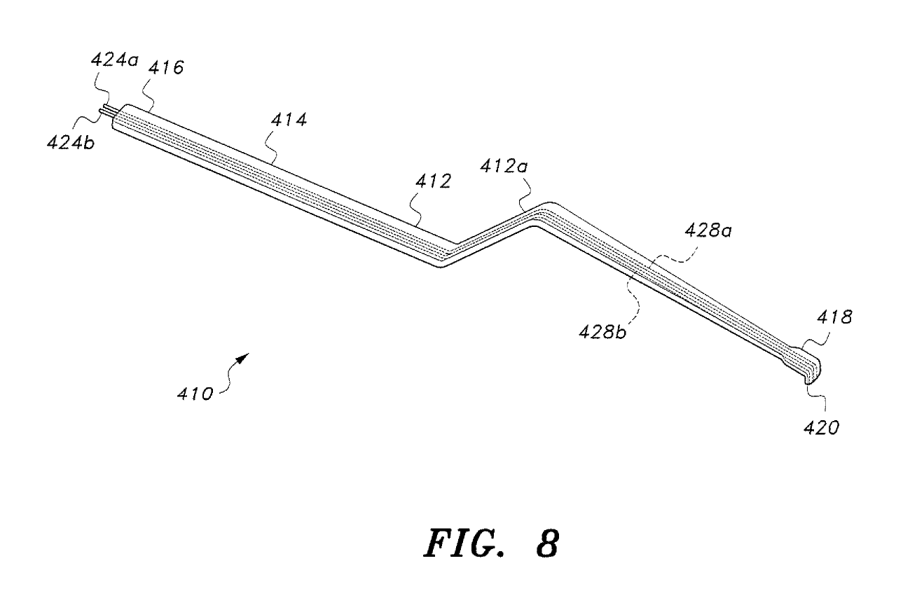

FIG. 8 provides a perspective view of another alternative embodiment of the nerve root retractor, designated as nerve root retractor or surgical implement or instrument 410. The nerve root retractor 410 of FIG. 8 includes an elongate shank 412 having a handle portion 414 and proximal end 416, and a distal tip portion 418 with distal inlet and outlet end 420 opposite the handle portion 414 and end 416. The surgical implement 410 of FIG. 8 does not include a separate handle formed about the handle portion 414 of the tool. However, it will be seen that such a handle (e.g., handle 22 of the implement 10 of FIG. 1, or handle 122 of the implement 110 of FIG. 5) may be provided with the implement 410, if so desired. Alternatively, the handles shown in other embodiments may be deleted from those implements, if so desired.

Separate irrigation and suction passages, respectively 428a and 428b, extend through the length of the shank 412, as in the cases of the retractors 110 through 310 respectively of FIGS. 5 through 7. The irrigation and suction passages 428a, 428b enter the instrument 410 through separate inlet and outlet fittings, respectively 424a and 424b, essentially as in the instrument 110 of FIG. 5. The primary difference between the retractor 410 of FIG. 8 and the retractor 310 of FIG. 7 is that the shank 412 includes a medial offset portion 412a therein, between the handle portion 414 and the distal end 418. The offset portion 412a may include bends of about 30 degrees at each end thereof, or bends of other angles as desired. The distal tip portion 418 and the extreme distal inlet and outlet end 420 of the surgical implement 410 of FIG. 8 will also be seen to differ somewhat from certain other embodiments, with the tip portion 410 being slightly widened and flattened and the inlet and outlet end 420 having an approximately 90 degree bend therein. Again, these shapes and angles may be adjusted as desired.

In conclusion, the periosteal elevator and nerve root retractor surgical instruments of the present invention greatly facilitate various surgical procedures by eliminating the need for additional instruments performing solely irrigation and suction tasks. This greatly simplifies the surgical environment by reducing the number of implements in the surgical wound. While a periosteal elevator and various embodiments of a nerve root retractor are disclosed herein, it will be seen that the placement of irrigation and suction passages within the shanks, shafts, or stems of other surgical instruments may be achieved as well, with the present disclosure encompassing such additional instruments having suction and irrigation passages formed integrally therein.

It is to be understood that the present invention is not limited to the embodiments described above, but encompasses any and all embodiments within the scope of the following claims.

I claim:

1. A surgical implement with suction and irrigation passages, comprising:
an elongate shank having a handle portion, a handle end, and a distal tip portion having a distal inlet and outlet end opposite the handle portion;
an irrigation passage disposed within and extending through the shank from the handle end to the distal tip portion of the shank, the irrigation passage being integrally formed within the shank; and
a suction passage disposed within and extending through the shank from the handle end to the distal tip portion of the shank, the suction passage being integrally formed within the shank.

2. The surgical implement with suction and irrigation passages according to claim 1, wherein the implement is a periosteal elevator having a broad and thin tip portion.

3. The surgical implement with suction and irrigation passages according to claim 1, further comprising:
a plenum disposed in the distal tip portion; and
a plurality of distal suction and irrigation passages extending from the plenum to the distal inlet and outlet end.

4. The surgical implement with suction and irrigation passages according to claim 1, wherein the shank is straight.

5. The surgical implement with suction and irrigation passages according to claim 1, wherein the shank has at least one bend therein.

6. The surgical implement with suction and irrigation passages according to claim 1, wherein the shank is formed of surgical grade corrosion-resistant steel.

7. The surgical implement with suction and irrigation passages according to claim 1, wherein the shank is formed of at least one material selected from the group consisting of plastic and carbon fiber.

8. The surgical implement with suction and irrigation passages according to claim 1, further comprising a handle disposed upon the handle portion of the shank.

9. A surgical implement with suction and irrigation passages, comprising:
a periosteal elevator having an elongate shank defining a handle portion, a handle end, and a distal tip portion having a distal edge opposite the handle end;
a plenum disposed within the distal tip portion of the shank;
an irrigation passage disposed within and extending through the shank from the handle end to the plenum, the irrigation passage being integrally formed within the shank;
a suction passage disposed within and extending through the shank from the handle end to the plenum, the suction passage being integrally formed within the shank; and
a plurality of common passages extending from the plenum to the distal edge of the distal tip portion.

10. The surgical implement with suction and irrigation passages according to claim 9, wherein the distal tip portion smoothly tapers from the shank to a broad, flat, and thin distal edge.

11. The surgical implement with suction and irrigation passages according to claim 9, wherein the shank is straight.

12. The surgical implement with suction and irrigation passages according to claim 9, wherein the shank has at least one bend therein.

13. The surgical implement with suction and irrigation passages according to claim 9, wherein the shank is formed of at least one material selected from the group consisting of surgical grade corrosion-resistant steel, plastic, and carbon fiber.

14. The surgical implement with suction and irrigation passages according to claim 9, further comprising a handle disposed upon the handle portion of the shank.

15. A surgical implement with suction and irrigation passages, comprising:
a nerve root retractor having an elongate shank defining a handle portion, a handle end, and a distal tip portion having a distal inlet and outlet end opposite the handle end;
an irrigation passage disposed within and extending through the shank from the handle end to the distal inlet and outlet end thereof, the irrigation passage being integrally formed within the shank; and
a suction passage disposed within and extending through the shank from the handle end to the distal inlet and outlet end thereof, the suction passage being integrally formed within the shank.

16. The surgical implement with suction and irrigation passages according to claim 15, wherein the shank is straight.

17. The surgical implement with suction and irrigation passages according to claim 15, wherein the shank has at least one bend therein.

18. The surgical implement with suction and irrigation passages according to claim 15, wherein the shank is formed of surgical grade corrosion-resistant steel.

19. The surgical implement with suction and irrigation passages according to claim 15, wherein the shank is formed of at least one material selected from the group consisting of plastic and carbon fiber.

20. The surgical implement with suction and irrigation passages according to claim 15, further comprising a handle disposed upon the handle portion of the shank.
